(19) United States
(12) Patent Application Publication
(10) Pub. No.: US 2007/0275400 A1
(43) Pub. Date: Nov. 29, 2007

(54) Multivariate Random Search Method With Multiple Starts and Early Stop For Identification Of Differentially Expressed Genes Based On Microarray Data

(76) Inventors: Ashot Chilingarian, Yerevan (AM); Aniko Szabo, Layton, UT (US); David Jones, Salt Lake City, UT (US)

Correspondence Address: NEEDLE & ROSENBERG, P.C., SUITE 1000, 999 PEACHTREE STREET, ATLANTA, GA, 30309-3915, US

(21) Appl. No.: 11/754,950

(22) Filed: May 29, 2007

Related U.S. Application Data
(63) Continuation of application No. 10/506,409, filed on Feb. 15, 2006, now abandoned.
(60) Provisional application No. 60/361,068, filed on Mar. 1, 2002.

(51) Int. Cl. C12Q 1/68

(52) U.S. Cl. 435/6

(57) ABSTRACT

The present invention provides multivariate methods for analyzing microarray gene expression data of high dimensional space and thereby identifying differentially expressed genes. The methods of this invention provide a random search procedure with multiple starts and early stop. Larger sets of differentially expressed genes may be identified using the methods of this invention starting from feature spaces of smaller dimensionality where accurate estimates on covariance matrix can be made.

### BACKGROUND OF THE INVENTION

1. Field of the Invention

The present invention relates in general to statistical analysis of microarray data generated from nucleotide arrays. Specifically, the present invention relates to identification of differentially expressed genes by multivariate microarray data analysis. More specifically, the present invention provides an improved multivariate random search method for identifying large sets of genes that are differentially expressed under a given biological state or at a given biological locale of interest. The method of the invention implements multiple starts and early stop in the random search of sets of differentially expressed genes.

2. Description of the Related Art

Gene expression analyses based on microarray data promises to open new avenues for researchers to unravel the functions and interactions of genes in various biological pathways and, ultimately, to uncover the mechanisms of life in diversified species. A significant objective in such expression analyses is to identify genes that are differentially expressed in different cells, tissues, organs of interest or at different biological states. So identified, a set of differentially expressed genes associated with a certain biological state, e.g., tumor or certain pathology, may point to the cause of such tumor or pathology, and thereby shed light on the search of potential cures.

In practice, however, gene expression studies are hampered by many difficulties. For example, poor reproducibility in microarray readings can obscure actual differences between normal and pathological cells or create false positives and false negatives. The tension between the extremely large number of genes present (hence high dimensionality of the feature space) and the relatively small number of measurements also poses serious challenges to researchers in making accurate diagnostic inferences.

Existing methods for selecting differentially expressed genes are typically univariate, not taking into account the information on interactions among genes. As appreciated by an ordinary skilled molecular biologist, genes do not operate in isolation—activation of one gene may trigger changes in the expression levels of other genes. That is, genes may be involved in one or more pathways. Therefore, determination of differentially expressed genes calls for consideration of covariance structure of the microarray data, in addition to, for example, mean expression levels. In this regard, however, application of well-established statistical techniques for multidimensional variable selection encounters much difficulty. This is so because, in one aspect, the small number of independent samples and the presence of outliers make the estimates on selected variables unstable for large dimensions. In other words, only small sets of genes can be meaningfully considered while a relatively large number of genes are potentially differentially expressed. It is generally impossible to compare all gene subsets and find the optimal one because the number of possible gene combinations is prohibitively large. On the other hand, if a global optimum could be found, it might be overly specific to a training sample due to overfitting. Thus, it remains a significant challenge to scale methods for identifying differentially expressed genes to deal with microarray data of high dimensional space.

Therefore, there is a need to address the difficulties in applying multivariate analysis to microarray data—a need to establish rigorous methods for identification of differentially expressed genes from high dimensional gene expression data.

### SUMMARY OF THE INVENTION

It is therefore an object of this invention to provide multivariate methods for analyzing microarray gene expression data of high dimensional space and thereby identifying differentially expressed genes. Particularly, it is an object of this invention to provide methods for identifying larger sets of differentially expressed genes starting from feature spaces of smaller dimensionality where accurate estimates on covariance matrix can be made. More particularly, the present invention provides a random search method with multiple starts and early stop.

In accordance with the present invention, there is provided methods for identifying a set of genes from a multiplicity of genes whose expression levels at a first and a second state, in a first and a second tissue, or in a first and a second types of cells are measured in replicates using one or more nucleotide arrays, thereby generating a first plurality of independent measurements of the expression levels for the first state, tissue, or type of cells and a second plurality of independent measurements of the expression levels for the second state, tissue, or type of cells. The method comprises, (a) identifying a quality function capable of evaluating the distinctiveness between the first plurality and the second plurality; (b) selecting a subset of genes, whose expression levels in the first and second states, tissues, or types of cells are represented in the first plurality and the second plurality, respectively; (c) calculating the values of the quality function for the subset of genes in the first state and said second state based on the first and second plurality, thereby determining the distinctiveness of the first and the second plurality; (d) substituting a gene in the subset with one outside of the subset, thereby generating a new subset, and repeating step (c), keeping the new subset if the distinctiveness increases and the original subset if otherwise; (e) repeating steps (c) and (d) for a first predetermined number of times, thereby identifying a locally optimal subset of genes; (f) repeating steps (b) to (e) for a second predetermined number of times, thereby identifying the second predetermined number of the locally optimal subsets; and (g) integrating the second predetermined number of the locally optimal subsets into the set of genes, wherein the set is larger than the locally optimal subsets in size.

According to the present invention, in certain embodiments, the states may be biological states, physiological states, pathological states, and prognostic states. In other embodiments, the tissues may be normal lung tissues, cancer lung tissues, normal heart tissues, pathological heart tissues, normal and abnormal colon tissues, normal and abnormal renal tissues, normal and abnormal prostate tissues, and normal and abnormal breast tissues. In yet other embodiments, the types of cells may be normal lung cells, cancer lung cells, normal heart cells, pathological heart cells, normal and abnormal colon cells, normal and abnormal renal cells, normal and abnormal prostate cells, and normal and abnormal breast cells. In still other embodiments, the types of cells may be cultured cells and cells isolated from an organism.

According to another embodiment of this invention, the integrating is performed by selecting the genes whose frequency of occurrences in the second predetermined number of the locally optimal subsets exceeds a third predetermined number. In certain embodiments, the third predetermined number is 1% or 5%. According to yet another embodiment, the first predetermined number is sufficiently small such that the global maximum is not reached. According to still another embodiment, the quality function is a parametric function or a non-parametric function. In a further embodiment, the parametric function is selected from the group consisting of the Mahalanobis distance and the Bhattacharya distance.

In various embodiments of the invention, the nucleotide arrays may be arrays having spotted thereon cDNA sequences and/or arrays having synthesized thereon oligonucleotides.

### BRIEF DESCRIPTION OF DRAWINGS

FIG. 1 depicts the steps of multivariate random search with multiple starts and early stop according to one embodiment of the invention.

FIG. 2 shows the differences of gene selection using multivariate random search with early or late stop according to various embodiments of the invention. First row are histograms of the values from the “last best iteration” in the N_{cycle} search. Second row are histograms of the estimated Mahalanobis distances for the N_{cycle} selected sets. Third row are histograms of the frequency of occurrences of the differentially expressed genes (1-20) in one of the selected sets.

FIG. 3 shows ROC curves for various values of Nᵢₜₑᵣ controlling the stopping time based on 10 simulated data sets, error bars depicting the corresponding standard errors.

FIG. 4 shows the differences of gene selection from same or different tissues using multivariate random search with early or late stop according to various embodiments of the invention. First row are histograms of the values of the “last best iteration” in the N_{cycle} searches. Second row are histograms of the estimated Mahalanobis distances for the N_{cycle} sub-optimal sets.

FIG. 5 shows the differences of the frequency of inclusion in the selected locally optimal set using multivariate random search according to one embodiment of the invention, applied to same or different tissue samples and with or without controls.

### DETAIL DESCRIPTIONS OF DISCLOSURE

Definition

As used herein the term “microarray” refers to nucleotide arrays; “array,” “slide,” and “chip” are used interchangeably in this disclosure. Various kinds of nucleotide arrays are made in research and manufacturing facilities worldwide, some of which are available commercially. There are, for example, two kinds of arrays depending on the ways in which the nucleic acid materials are spotted onto the array substrate: oligonucleotide arrays and cDNA arrays. One of the most widely used oligonucleotide arrays is GeneChip™ made by Affymetrix, Inc. The oligonucleotide probes that are 20- or 25-base long are synthesized in silico on the array substrate. These arrays tend to achieve high densities (e.g., more than 40,000 genes per cm²). The cDNA arrays, on the other hand, tend to have lower densities, but the cDNA probes are typically much longer than 20- or 25-mers. A representative of cDNA arrays is LifeArray made by Incyte Genomics. Pre-synthesized and amplified cDNA sequences are attached to the substrate of these kinds of arrays.

Microarray data, as used herein, encompasses any data generated using various nucleotide arrays, including but not limited to those described above. Typically, microarray data includes collections of gene expression levels measured using nucleotide arrays on biological samples of different biological sates and origins. The methods of the present invention may be employed to analyze any microarray data; irrespective of the particular microarray platform from which the data are generated.

Gene expression, as used herein, refers to the transcription of DNA sequences, which encode certain proteins or regulatory functions, into RNA molecules. The expression level of a given gene refers to the amount of RNA transcribed therefrom measured on a relevant or absolute quantitative scale. The measurement can be, for example, an optic density value of a fluorescent or radioactive signal, on a blot or a microarray image. Differential expression, as used herein, means that the expression levels of certain genes are different in different states, tissues, or type of cells, according to a predetermined standard. Such standard maybe determined based on the context of the expression experiments, the biological properties of the genes under study, and/or certain statistical significance criteria.

The terms “vector,” “probability distance,” “distance,” “the Mahalanobis distance,” “the Euclidean distance,” “feature,” “feature space,” “dimension,” “space,” “type I error,” “type II error,” and “ROC curve” are to be understood consistently with their typical meanings established in the relevant art, i.e. the art of mathematics, statistics, and any area related thereto. For example, a set of microarray data on p distinct genes represents a random vector X=X₁, . . . , Xₚ with mutually dependent components.

Improved Random Search Procedure with Multiple Starts and Early Stop

Random search algorithms have been used for finding optima in complex combinatorial spaces. See, e.g., Zhigljavsky A A., Vol. 65, Mathematics and its Applications, Kluwer Academic Publishers Group, Dordrecht, 1991. The improved random search procedure according to one embodiment of this invention applies a local search procedure multiple times and then integrates the selected sets of genes to build a global optimal set of differential expressed genes. To prevent overfitting, short local searches may be performed. Local maximum regions are carefully examined and convergence to a unique global maximum is avoided. The method can be applied in conjunction with a variety of parametric and non-parametric quality functions, which are discussed in more detail in the next section. In certain embodiments, the improved random search procedure with multiple starts and early stop includes the following steps:

1. Randomly select N_{subset} genes from Nₐₗₗ, wherein N_{subset} is the number of genes in a subset, Nₐₗₗ is the total number of the genes, and N_{subset} is smaller then Nₐₗₗ.

2. Evaluate the quality function for the N_{subset} genes.

3. Generate a new evaluation point (i.e., starting point) by swapping one or more randomly selected genes between the currently selected set and the rest of the genes, thereby identifying a new N_{subset}.

4. Evaluate the quality function for the new N_{subset} genes; if its value has decreased, then return to the previous N_{subset}, otherwise maintain the new N_{subset}.

5. Repeat steps 3 and 4 until the number of iterations reaches a predetermined number—let it be Nᵢₜₑᵣ—then save the resultant N_{subset} at that point.

6. Repeat steps 1-5 N_{cycle} times.

7. Evaluate the resultant N_{cycle} groups of N_{subset} genes to identify an integrated larger set of genes.

In step 7, a post-processing step, the local optima are combined to provide a final, global solution, i.e., an integrated larger set of differentially expressed genes. Heuristically, strongly differentially expressed genes should appear in many of the local maxima. Therefore, each gene to be included in the final set of differentially may be identified based on the frequency of its occurrence in the sub-optimal (i.e., locally optimal) sets derived from each of the N_{cycle} cycles, as performed in steps 1-6 above. A conservative estimate of the p-value corresponding to the observed frequency can be calculated. For example, if a gene is not differentially expressed, the probability that it will be in the selected subset by chance is expected to be equal to N_{subset}/Nₐₗₗ, and most likely smaller. As the number of repetitions N_{cycle} is large, the final selection frequency of this gene may be approximated by a Poisson distribution with a mean N_{cycle} *N_{subset}/Nₐₗₗ. Based on this null-distribution the corresponding p-values for each gene may be calculated.

Generally, N_{subset} is limited by the number of available training samples (e.g., the number of microarray slides in a typical experiment) and hence, N_{subset} may be significantly smaller than Nₐₗₗ. Depending upon the particular quality function of choice, the nature and the extent of this limitation may vary; but, generally, both parametric and non-parametric criteria are sensitive to the scarceness of training samples in a high-dimensional feature space. In this connection, one significant advantage of the improved random search method disclosed herein is that, the detectable number of the differentially expressed genes is not limited by N_{subset}, even though the depth of the estimated interaction structure (e.g., the covariance matrix) may be affected. In other words, a relatively large set of differentially-expressed genes may be identified by integrating the subsets of genes selected from multiple local searches. In some embodiments of this invention, the final set of differentially expressed genes is significantly larger in size than the subset identified in the local search, i.e., the locally optimal subset: N_{subset}.

The determination of Nᵢₜₑᵣ is crucial for preventing overfitting. It cannot be too small because a small value may not permit finding truly differentially expressed genes. On the other hand, too large a number will not be efficient. When the value is too big, the same maximum may be attained in many iterations of search because of overfitting.

With regard to N_{cycle}, this is a number that substantiates the variability of this random search procedure. It may be as large as possible, only limited by the applicable CPU power (e.g., N_{cycle}=1,000,000 may be used).

Quality Functions Used in Conjunction with the Random Search Procedure

A variety of quality functions may be used in conjunction with the improved random search procedure in various embodiments of this invention. A quality function measures the “distinctiveness” of the two tissues or two biological states under comparison based on a set of genes, taking into account the correlation structure. Generally, properly specified parametric methods are more powerful than non-parametric methods due to the utilization of additional information accounted in the model, although such parametric quality functions nay be sensitive to any departure from the model. With microarray data, since small sample sizes are a prevalent problem, choosing an appropriate parametric quality function may be advantageous in its power, whereas a non-parametric random search method may be more robust. One parametric measure of the differences between two multidimensional sample is the Mahalanobis distance, which is used in one embodiment of this invention. See Mahalanobis P C., Proceedings of the National Institute of India (1936) 2 Vol. 49.

$${R}_{Mah}^{2} = {( v - u )}^{′} {( \frac{{\sum }_{u} + {\sum }_{v}}{2} )}^{- 1} ( v - u ) ,$$

where v and u are the sample means and Σᵤ, Σᵥ are the two sample variance-covariance matrices. It is a natural extension of the t-statistic to a multidimensional setting. Because of the matrix inverse involved, the calculation of the Mahalanobis distance at every step of the search—for N_{cycle}·Nᵢₜₑᵣ times—may appear to be prohibitive. However, with the improved random search procedure of this invention, changes in the vectors are only in one dimension on every step (see supra, steps 1-5); therefore, a fast update formula may be permitted. See, e.g., McLachlan G J., Discriminant Analysis and Statistical Pattern Recognition, (1992) Wiley, N.Y.

In another embodiment of this invention, the Bhattacharya distance may be used, especially where differences in both the mean and the covariance structure are of interest.

$${R}_{Bha}^{2} = \frac{1}{8} {R}_{Mah}^{2} + \frac{1}{2} ln \frac{\vert \frac{{\sum }_{u} + {\sum }_{v}}{2} \vert}{\sqrt{\vert {\sum }_{u} \vert \vert {\sum }_{v} \vert}} .$$

p Similarly, other parametric or non-parametric dissimilarity measures may be used in various alternative embodiments in conjunction with the improved random search procedure disclosed herein. Such different choices of quality functions each may be designed to deal with microarray data with different characteristics.

Further, when using various quality functions, various background reduction, normalization, and other adjustment procedures may be applied to the microarray data. For examples rank-based adjustment and the typical mean-log adjustment (dividing by mean and take logarithm) may be used. In one embodiment, the following adjustment is implemented: the data points on each slide or array were replaced by their normal scores using the formula
x=Φ⁻¹(rankⱼxᵢⱼ/Nₐₗₗ),
where Φ⁻¹ (α) is the 100α^{th} percentile of the standard normal distribution and rankⱼxᵢⱼ is the rank of Xᵢⱼ among all of the observations on the j^{th} slide. See, Tsodikov A. et al., (2002) Bioinformatics 18:251-260.
Computer Simulation of the Multivariate Search Method

A simulation study was performed to evaluate the improved random search method. Totally 1000 genes were divided into subsets of equal size 20. One of the subsets was selected to be deemed as differentially expressed with the gene-specific ratio d randomly generated for each of the genes from a lognormal distribution with mean 1 and variance 0.5. The correlation structure was kept the same in the two hypothetic tissues. In the selected subset some of the genes exhibited large over- or under-expression, while others with d˜1 changed their expression level only slightly. The simulation was performed on 20 slides or arrays with one of the tissues on the green channel and the other on the red channel. The relevant parameters for the random search were set: N_{cycle}=10,000, N_{subset}5; and, the Mahalanobis distance was used as the quality function.

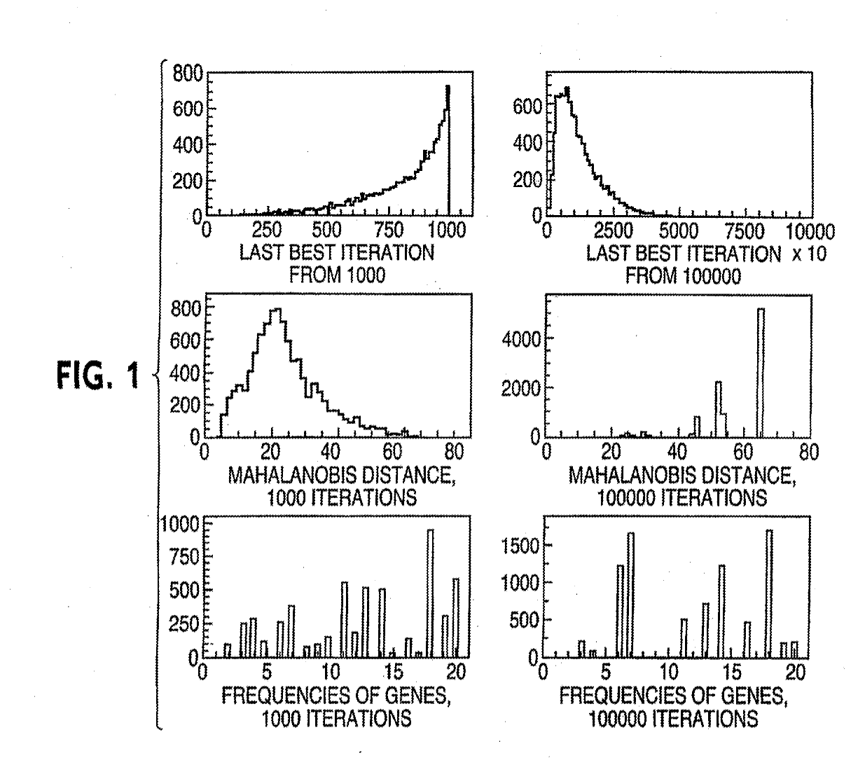

Referring to FIG. 1 the results of the random search procedure are compared between Nᵢₜₑᵣ=1000 (in the left panels) and Nᵢₜₑᵣ=100,000 (in the right panels). The two graphs on the top show the histograms of the values of the “last good iteration”—the number of iterations after which no new successful steps were encountered (i.e., when no new subset was found any more at step 4 of the aforementioned procedure and thus the final set was determined). The two histograms demonstrate that 1000 iterations were a little less than sufficient to reach the global maximum, whereas 10,000 iterations were more than enough for the random search to converge.

The middle graphs illustrate the same phenomenon in another way. In the case of early stopping, i.e., when Nᵢₜₑᵣ=1000, the distribution of the Mahalanobis distances corresponding to the N_{cycle} sub-optimal sets is unimodal with high variability. Thus, the procedure has explored many different local maxima with a variety of corresponding values of the quality function. On the other hand, when the number of iterations increase, e.g., when Nᵢₜₑᵣ=100,000, the distribution of the Mahalanobis distances achieved in the sub optimal sets became very discrete. In about half of the cases the search reached the global maximum on a unique combination of genes. Therefore, in this situation, although the global maximum was found, many local maxima and the corresponding differentially expressed genes from the various subsets were missed. When early stop is carried out at the 1000-th iteration, none of 10,000 cycles found the global maximum, but a variety of genes were selected.

At the bottom panel of FIG. 1, the frequencies of selection for the 20 genes in the differentially expressed gene set are plotted. The x-axis represents the number of the genes: from gene No. 1 to No. 20. With Nᵢₜₑᵣ=1,000, i.e., when the early stop was implemented, 17 from 20 genes pass the selection criteria (predetermined to be a frequency of occurrence higher than 0.5%). With Nᵢₜₑᵣ=100,000, i.e., when the early stop was not implemented, only 10 genes met the 0.5% frequency standard when the global maximum was attained.

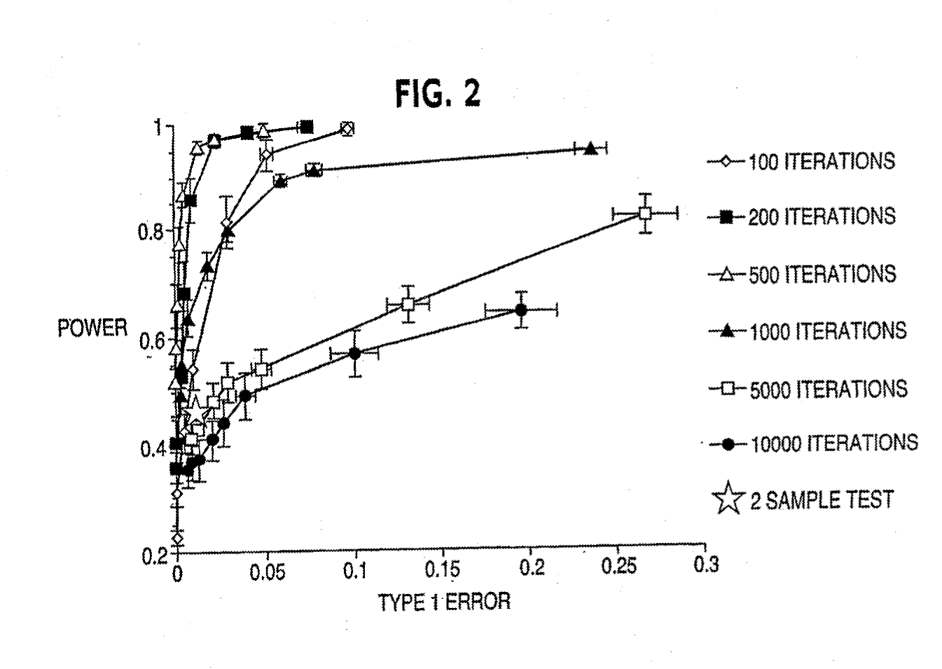

Referring to FIG. 2 the ROC curves corresponding to values of Nᵢₜₑᵣ ranging from 100 to 10,000 based on 10 independently simulated data sets were plotted. Other parameters were held constant, that is, N_{subset}=5, N_{cycle}=10,000. For each search, a list of genes with associated frequencies of occurrence in the selected subsets were complied and a final set of differentially expressed genes was identified by applying cutoff values ranging from 0.1% to 10% in frequency. Based on the null hypotheses of no differential expression, for each of these sets, the ratio of type I error (i.e., the false positive) was defined as the proportion of non-differentially expressed genes that was selected into the final set. And the ratio of the type II error (i.e., the false negative) was defined as the proportion of the genes in the differentially expressed subsets that were not included in the final set. The resulting ROC curves are shown in FIG. 2. Also, as a reference, the point representing the type I error and the power of the marginal t-test with 5% significance level is also plotted (referring to the star in FIG. 2). Comparing the ROC curves in FIG. 2, a skilled artisan can note that the value of Nᵢₜₑᵣ significantly affects the performance of the random search procedure: long searches are inferior to searches with early stop. There ought to be, however, a limit on bow early the search should stop, because very short searches are not likely to reach any local maxima. According to FIG. 2, the best performance was achieved when Nᵢₜₑᵣ=500.

The invention is further described by the following examples, which are illustrative of the invention but do not limit the invention in any manner.

### EXAMPLE 1

### A Detailed Illustration of Random Search with Multiple-Starts and Early Stop

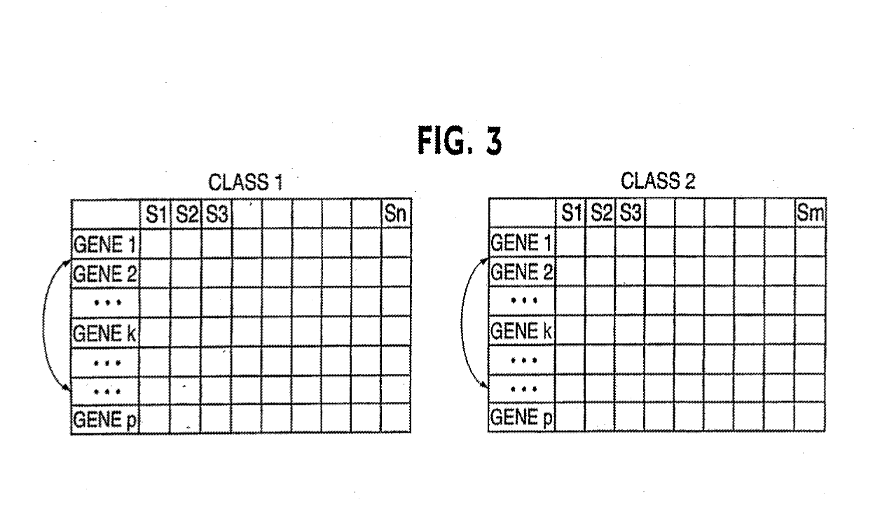

Referring to FIG. 3, suppose there are p genes and n and m independent samples in the two classes respectively, this procedure finds a group of genes differentially expressed in these classes using information on the k-variate dependence structure.

1. Repeat the following Nᵢₜₑᵣ times. Nᵢₜₑᵣ is not too large; early stop—stop before convergence—is implemented.
a. Randomly select k genes (genes 2 to gene k in FIG. 3) that will serve as the seed of the random search.
b. Calculate the distance between the two classes based on the k initially selected genes.
c. Randomly select a gene (e.g., gene 2 in FIG. 3) from the current gene set (gene 2 to gene k in FIG. 3), remove it from the set and replace it with a gene randomly selected from outside of the set (e.g., any of gene k+1 to gene p in FIG. 3, let it be gene x).
d. Calculate the distance between the two classes based on the new gene set (gene 3 to gene k, plus gene x). If this distance is larger than the previously calculated one, then keep the change, otherwise revert to the previous set.
e. Retain the selected sub optimal set of genes, i.e., the set that has the largest distances between the two classes.

2. Repeat step 1 N_{cycle} times, obtain N_{cycle} sets of genes of size k.

3. For each gene, calculate the frequency of its occurrence as a member of a sub-optimal set.

4. The final set of genes is defined as the genes that have a frequency of occurrence exceeding a preset limit.

### EXAMPLE 2

### A Source Code Segment Implementing Random Search with Multiple Starts and Early Stop—Step 1 and 2 of Example 1

Program gene1
c
parameter (nall=1000, ncl=10, niter=500, m=20,l=2,nt=2)
parameter (ishift=3000,NCYCLE=1000)
parameter (genadd=5.,disp=1.,debug=2.)
parameter (expmax=20.,strang=1.e−15)
parameter (kcl=5,iap=1,nex=10)
parameter (pat=1.5,dpat=0.,frailty=0.2,ncls=20,purity=0.85)
c
CHARACTER*50 jmode,qualit, ranf,ku,stat,start,normal,mixup
CHARACTER*50 sound,ill
DIMENSION AP(L*IAP),DEL(M*1)
DIMENSION DEN((KCL+2)*L),PST(L),DFM(L*(KCL+2)*L*iap)
DIMENSION F(KCL+2),DS(M*L*L*(KCL+2))
DIMENSION DI(ncl),DETER(L),rank1(m),rank2(m)
c
dimension err(kcl+2),g((kcl+2)*1),ent(1)
c
Dimension inum(ncl),b(nall*m*l),a(nall*m*l),cl(ncl*m*l),u(m*l)
dimension e(ncl*ncl),ito(1),ind3(niter)
dimension e1(ncl*ncl),e2(ncl*ncl),e3(ncl*ncl),z(nex*nex)
dimension imbest(ncl),x(m*l),v(nall),m22(m*1),ind2(nall)
dimension r(ncl*ncl*l),r2(ncl*l),r3(ncl*ncl*l)
dimension mv(kcl),ff(kcl),dd(kcl),rr(kcl)
dimension stud(nall), tkolm(nall),tmann(nall)
dimension iex(nex)
c
character*10 ndata, ntime
data iex/1,2,3,4,5,6,7,8,9,10/
data f/0.5,0.6,0.7,0.8,0.9,1.0,1.1/
data ap/0.5,0.5/
data qualit /‘mahalo’/
data jmode /‘one-leave-out-’/
data mixup /‘no’/
data ranf /‘ffile’/
data normal/’gauss’/
data stat /‘param’/
data start /‘bestcor’/
c
sound=‘red1.txt’
ill=‘red2.txt’
c
OPEN (unit=NT,FILE=‘b’,
FORM=‘FORMATTED’,STATUS=‘unknown’)
open(unit=11,file=sound,form=‘formatted’,
* status=‘old’)
open(unit=22,file=ill,form=‘formatted’,
* status=‘old’)
open(unit=68,file=‘inbest.dat’,form=‘formatted’,
* status=‘unknown’)
open(unit=69,file=‘best.dat’,form=‘formatted’,
* status=‘unknown’)
c
write(nt,‘(//30x,“GENE CLUSTER MASTER”/)’)
write(nt,‘(“Number of slides M = ”, i3)’)m
write (nt,‘(“Number of genes NALL =”,i5,
* “ ,genuin cluster size=”,i4,“ ,to be searched:”,i4)’)
* nall,ncls,ncl
write (nt,‘(“DATA normalization to(by) = ”,A10)’)normal
write (nt,‘(“Type of Statistics Used = ”,A10)’)stat
if(ranf.ne.‘ffile’)then
write(nt,‘(“Overexpression of Poisoned Genes ”,f5.1,
* “ Variance ”,f5.1)’)
* genadd,disp
write(nt,‘(“Random Numbers Generator ”,a10,” ,Shift”,i5)’)
*ranf,ishift
end if
write(nt,‘(//30x,”SIMULATION PARAMETERS”/)’)
write(nt,‘(“Sound data from: ”a30)’)sound
write(nt,‘(“Patology data from: ”a30)’)ill
write(nt,‘(“SIMULATED PATOLOGY LEVEL: ”,f3.1,“+/−”,f3.1)’)
* pat,dpat
write(nt,‘(“Level of mutual Frailty for Cluster: ”, f5.2)’)
* frailty
write(nt,‘(“Mixture: ”,f5.2,“LogNorm +”,f5.2”Uniform”)’)
* purity,1.˜purity
c
write(nt,‘(//30x,“SEARCH PARAMETERS”/)’)
write(nt,‘(”MIXUP the GENES? ”, a10)’)mixup
write(nt,‘(”SEARCH MODE ”, a10)’)qualit
write(nt,‘(“Number of Random Search Trials:”i7)’)
* niter
if (nex.ne.0) then
write(nt,‘(”ATTENSION!, Genes Excluded:”/10(10i6/))’)
* (iex(i),i=1,nex)
end if
if(qualit.eq.‘parz’.or.qualit.eq.‘knn’)then
write(nt,‘(“MODE OF BAYES QUALITY ”, a10)’)ku
write (nt,‘(“Number and values of kernels”,i5/
* 15 f5.1)’)kcl,(f(i),i=1,kcl)
end if
do i=1,ishift
aa=rndm(−1.)
end do
c
if(ranf.eq.‘uni’)then
do i=1,nall*m*2
b(i)=1.+rndm(−1.)*disp
end do
c
else if (ranf.eq.‘normco’)then
do i=1,nall*m−1,2
call normco(b(i),b(i+1),5.,3.,disp,disp,0.9)
end do
do i=nall*m+1,nall*m*2−1,2
call normco(b(i),b(i+1),5.,3.,disp,disp,−0.9)
end do
else if(ranf.eq.‘ffile’) then
call rfromf(b, nall,m,l)
c
else
write(nt,‘(“no such data mode”,a10)’)ranf
stop 67
end if
if(ranf.ne.‘ffile’) then
c
do j=m,2*m−1
do i=nall*j+1,nall*j+ncl
b(i)=b(i)+genadd
end do
end do
if (nall.le.10) then
write (nt,‘(10f7.2/)’)b
end if
end if
c
if(mixup.eq.’yes’) then
do i=1,nall
ind2(i)=i
end do
do i =1, ishift
iin=rndm(−1.)*nall+1
iout=rndm(−1.)*nall+1
numold=ind2(iout)
ind2(iout)=ind2(iin)
ind2 (iin)=numold
if(iin.gt.nall.or.iout.gt.nall.or.iin.lt.l.or.iout.lt.1) then
write(*,‘(”BIGGGGG!!!!”, 3i18)’)i,iin,iout
end if
call exchange(b,nall,m,l,iin,iout,x,u)
end do
if (debug.ge.5) then
write (nt,‘(“Mixed Cluster”/1000(10i8/))’)
* (ind2(i),i=1,nall)
end if
end if
c
if (normal.ne.‘no’) then
call normalization(b,ind2,nall,m,l,stud,tkolm,normal)
end if
c
call tests(b,m22,ind2,nall,m,l,x,u,stud,tkolm,tmann,nt,ncl)
c
ito(1)=m
ito(2)=m
mb=ito(1)+ito(2)
istg=0
c
sd=0.
stiter=1.e20
c
do i=1,m*1
u(i)=1./m
end do
c
if(start.eq.‘bestcor’.and.nex.ge.2) then
do i=1,nex
inum(i)=iex(i)
end do
call assign(b,inum,cl,nex,nall,m,l)
c
c write (*,‘(“u(i) ”/10(10f8.5/))’)
c * (u(i), i=1,m*1)
c
call misr1(c1,r2,r,u,nex,ito,mb,l)
call covcr(r,r3,z,nex,m,l)
write (nt,‘(/25x,“CORRELATION MATRIX”/10(12i6/))’)
* (iex(i), i=1,nex)
write (nt,‘(/10(10f6.2/))’)
* (r3(i), i=1,2*nex*nex)
write (nt,‘(/25x,”FISHER MATRIX”/10(10i6/))’)
* (iex(i), i=1,nex)
write (nt,‘(/10(10f6.2/))’)
* (2(i), i=1,nex*nex)
write (nt,‘(/“Genes means ”/5(10f6.2/))’)
* (r2(i), i=1,2*nex)
c
call bhafas(r,r2,e,e1,e2,e3,rb,rm,rc,nex,qualit,debug)
write (nt,‘(/”Mahalonobis Distance: “,f12.2)’)rm
c
stop 777
end if
DO ICY=1,NCYCLE
ii=0
if(start.eq.‘random’) then
c
iin=rndm(−1.)*nall+1
inum(1)=iin
c
do i=2,ncl
88 continue
inew=rndm(−1.)*nall+1
do j=1,i−1
if(inew.eq.inum(i−j))then
go to 88
else
inum(i)=inew
end if
end do
end do
else if(start,eq.‘last’) then
DO I=1,NCL
ii=ii+1
inum(ii)=i+NALL−NCL
end do
else if(start.eq.‘first’) then
do i=nall, nall−ncl,−1
ii=ii+1
inum(ii)=i+NALL−NCL
end do
else if(start.eq.’frombest’) then
read(68,‘i7,e12.4,(10(10i6/))’)ll,qq,(inum(i),i=1,ncl)
else
stop 9999
end if
c
write (nt,‘(“ Initially Selected genes ”/
* 5(10i5/))’)inum
DO iter=1,niter
c
if (iter.ne.1)then
call change(inum,nall,ncl,iin,iout,numold,ind3,iter,niter,
* iex,nex)
else
iin=1
iout=1
numold=99
c
end if
if (iter.gt.1.and.iter.le.5.and.debug.ge.3.) then
write (nt,‘(“Iteration”,i4,“ Exchanged genes ”,3i5/
* “MASK Array”/
* 5(10i5/))’)iter,IIN,iout,numold,(inum(i),i=1,ncl)
end if
c
call assign(b,inum,cl,ncl,nall,m,l)
c
if(stat.eq.‘param’)then
call misr1(cl,r2,r,u,ncl,ito,mb,l)
c
if (debug.ge.3) then
write (nt,‘(“Genes cov ”/5(5f12.5/))’)r
call covcr(r,r3,z,ncl,m,l)
write (nt,‘(“Genes cor ”/5(5f12.5/))’)r3
write (nt,‘(“Genes means ”,5(5f12.2/))‘)r2
end if
else if (stat.eq.‘nonparam’)then
call SPIR1(cl,r2,R,X,V,NCL,M,L,m22,ind2,rank1,rank2)
if (debug.ge.5) then
write (nt,‘(“Genes spirmen ”/5(10f12.5/))’)r
write (nt,‘(“Genes medians ”,5(10f12.2/))’)r2
write (nt,‘(“Genes interQU ”,5(10f12.2/))’)
* (v(i),i=1,ncl*l)
c stop 777
end if
end if
c BHATTACHARYA DISTANCE
c
if(qualit.eq.‘bhata’) then
ss=rb
else if (qualit.eq.‘mahalo’) then
ss=rm
else if (qualit.eq.‘corcor’) then
ss=rc
c
else
write(nt,‘(“no such quality function”,a10)’) qualit
stop 67
end if
end if
c
IF(SS.GT.SD) THEN
SD=SS
ISTG=ISTG+1
c
c REMEMBERING OF BEST VALUES
c
c CALL UCOPY(inum,imbest,ncl)
do iu=1,ncl
imbest(iu)=inum(iu)
end do
ibest=iter
c
CALL DATIMH(NDATA,NTIME)
c
WRITE(*,‘(“SUCCESS at: ”,A12,2X,A12,
* “ ITERATION”,i7,“ QUALITY”,e14.6)’)
* NDATA,NTIME,ITER,SD
write(*,‘(10(10i5/))’) (inum(i),i=1,ncl)
if(debug.ge.2)then
WRITE(nt,‘(“GOOD!!!,Iteration and Q”,i7,e14.6)’)ITER,SD
write(nt,‘(10(10i5/))’) (inum(i),i=1,ncl)
end if
ELSE IF(SS.LE.SD) THEN
inum(iout)=numoId
if(debug.ge.3.and.iter.le.10)then
CALL DATIMH(NDATA,NTIME)
WRITE(nt,‘(“BAAD!!!,Qnew and Qbest”,i7,2e14.6)’)ITER,SS,SD
end if
END IF
if(SD.GT.STITER) then
write (NT,‘(“REQUIRED DISTANCE ACHIEVED!”,2e15.3)’)
* SD, STITER
go to 18
end if
c
END DO
18 continue
write(nt,‘(25x,“ CYCLE N ” i6/)’)ICY
write(nt,‘(“Distance used : ”,A6,“ Quality=“,e15.3)’)
* qualit,sd
write(nt,‘(“Number of successful steps : ”,i5)’)istg
write(nt,‘(“Best Cluster Obtained After: “,
* i9;20(10i7/))‘)
* niter,imbest
c rewind 68
write(69,‘(i7,f12.4,20i6)‘) ibest,sd,imbest
c write(69,‘(i7,f12.4)’) ibest,sd
END DO
c
stop
end
c
subroutine tests(b,m22,ind2,nall,m,l,x,u,stud,tkolm,tmann,nt,ncl)
dimension b(nall,m,l),stud(nall), tkolm(nall),tmann(nall)
dimension x(m*l),u(m*l),m22(m*l),ind2(nall)
i34=m*0.75
i14=m*0.25
i5=m*0.5+1
do i=1,nall
do j=1,m
x(j)=b(i,j,1)
u(j)=b(i,j,2)
end do
call sortzv(x,m22,m,1,1,0,0)
xd=(x(m22(i34))−x(m22(i14)))/1.35
xm=x(m22(i5))
call sortzv(u,m22,1,m,1,0,0)
ud=(u(m22(i34))−u(m22(i14)))/1.35
um=u(m22(i5))
stud(i)=abs(xm−um)/sqrt(xd*xd+ud*ud)
end do
call sortzv(stud,ind2,nall,1,1,0,0)
write (nt,‘(“N.Student Cluster”
*/1000(10i8/))’)
*(ind2(i),i=1,ncl)
call errors(ind2,nt,ncl,nall)
c
do i=1,nall
xm=0,
xm2=0,
um=0,
um2=0,
do j=1,m
x(j)=b(i,j,1)
u(j)=b(i,j,2)
end do
do j=1,m
xm=xm+x(j)
xm2=xm2+x(j)*x(j)
um=um+u(j)
um2=um2+u(j)*u(j)
end do
xm=xm/m
um=um/m
xd=xm2/m−xm*xm
ud=um2/m−um*um
stud(i)=abs(xm−um)/sqrt(xd+ud)
end do
call sortzv(stud,ind2,nall,1,1,0,0)
write (nt,‘(“Param Student Cluster”/1000(10i8/))’)
* (ind2(i),i=1,ncl)
call errors(ind2,nt,ncl,nall)
do i=1,nall
do j=1,m
x(j)=b(i,j,1)
x(j+m)=b(i,j,2)
end do
CALL UTEST(x,u,m,m,tmann(i),ZU,IERR)
end do
call sortzv(tmann,ind2,nall,1,0,0,0)
write (nt,‘(“Mann-Whitney Cluster”/1000(10i8/))’)
* (ind2(i),i=1,ncl)
call errors(ind2,nt,ncl,nall)
do i=1,nall
do j=1,m
x(j)=b(i,j,1)
u(j)=b(i,j,2)
end do
CALL kolm2(x,u,m,m,tkolm(i),Prob)
end do
call sortzv(tkolm,ind2,nall,1,1,0,0)
write (nt,‘(“Kolmogorov Cluster”/1000(10i8/))’)
* (ind2(i),i=1,ncl)
call errors(ind2,nt,ncl,nall)
return
end
c

### EXAMPLE 3

### A Source Code Segment Implementing Integration of The Result from Local Searches to Build a Larger Set of Genes—Step 3 and Step 4 of Example 1

Program genecount
c
parameter (nall=1000, nclust=5, ntrial=10000,ncut=10,nr=22,nt=2)
parameter (nctrue=20,ipat=1,ntupw=1,ntidw=17,memw=100000)
parameter (debug=2.)
c
dimension a(nclust*ntrial),c(nall),cut(ncut),genprop(nclust)
dimension sel(nall)
dimension tontuple(nclust+3),ind(nall,nall),ind1(nall)
character*30 selgen
character*8 mode
data cut/0.000005,0.00001,0.00005,0.001,0.002,0.003,0.01,0.03,
* 0.05,0.08/
data cutpair/0.1/
data cpair/0.003/
data selgen /‘best.dat’/
data mode/‘sim’/
data niter /500000/
c
CHARACTER*1 opmo
CHARACTER*50 hbname
CHARACTER*8 tek(nclust+3)
DATA opmo/‘X’/,LRECLR/1024/,LRECLW/1024/
c
OPEN (UNIT=NT,FILE=‘b.count’,
FORM=‘FORMATTED’,STATUS=‘UNKNOWN’)
open(unit=nr,file=selgen,form=‘formatted’,status=‘old’)
c
hbname=‘genome.hbook’
tek(1)=’lastb’
tek(2)=’quality’
tek(3)=‘N_of_gen’
tek(4)=‘gene1’
tek(5)=‘gene2’
tek(6)=‘gene3’
tek(7)=‘gene4’
tek(8)=‘gene5’
c
c tek(i)=‘gene’//ichar(i−2)
c end do
if(ntupw.gt.0) then
call HROPEN(ntidw,‘ani98’,hbname,‘N’,Ireclw,ISTAT)
end if
call HBOOKN(ntidw,‘GENE SELECTION’,nclust+3,
* ‘//ani98’,memw,tek)
write(nt,‘(/10x,”GENE SORTER FOR ”, A10,
* “ EARLY STOP AT”,18)’)mode, NITER
qmean=0.
nmean=0
ntrj=0
ncount=1
do i=1,nclust
genprop(i)=0.
end do
do j=1,ntrial
read(22,*,err=100,end=99)nlast,quality,
* (a(i),i=ncount,ncount+nclust−1)
tontuple(1)=nlast
tontuple(2)=quality
jj=−1
kk=0
do i=4,nclust+3
jj=jj+1
tontuple(i)=a(ncount+jj)
if(tontuple(i).le.nctrue) then
genprop(i−3)=genprop(i−3)+1.
kk=kk+1
end if
end do
tontuple(3)=kk
call HFN(ntidw,tontuple)
ncount=ncount+nclust
ntrj=ntrj+1
nmean=nmean+nlast
qmean=qmean+quality
end do
go to 99
100 continue
write(*,’(”ERROR IN INPUT STREAM ON LINE: ”,i7)’)j
c
stop
c
99 continue
write (nt,‘(i7,“ Random Starts, Rm and Last ”,f12.4,i7)’)
* ntrj,qmean/ntrj,nmean/ntrj
c
if (mode.eq.‘sim’) then
write (nt,‘(/“% Of True”,10(5f12.4/))’)
* (genprop(i)/ntrj, i=1,nclust)
end if
call vzero(c,nall)
do j=1,nclust*ntrj
do k=1,nall
realk=real(k)
if(a(i).eq.realk) then
sel(k)=sel(k)+1
c(k)=c(k)+I./ntrj
end if
end do
end do
do j=1,ncut
do i=1,nall
ind1(j)=0
end do
ncount=0
do i=1,nall
if (c(i).gt.cut(j))Then
indI(i)=1
if (debug.ge.3) then
write(nt,‘(“GENE ”,I5,5x,
* “Appearance % ”,F12.5)’)I,C(I)
end if
ncount=ncount+1
END IF
end do
c
err1=0,
err2=0,
do i=1,nall
if(ind1(i).eq.1.and.I.Ie.nctrue)then
err1=err1+1,
else if(ind1(i).eq.1.and.i.gt.nctrue)then
err2=err2+1,
end if
end do
write(nt,‘(/“N of genes Selected with CUT ”,F9.5.,i8 )’)
* cut(j),ncount
if (mode.eq.‘sim’) then
write(nt,‘(“1 error: “,F9.5,”, 2 error”,F9.5)’)
* 1-err1/nctrue,err2/nall
write(nt,‘(“Eta = 1 − 1error/sqrt(2error): ”,F12.5)’)
* err1/nctrue/sqrt(err2/nall)
end if
end do
if (debug.ge.4.) then
ncount=0
do j=1,nall
do j=1,nall
ind(i,j)=0
end do
end do
do ni=1,ntrj
do j=1,nclust−I
k1=ifix(a(ncount+j))
do i=j+1,nclust
k2=ifix(a(ncount+i))
ind(k1,k2)=ind(k1,k2)+1
end do
end do
ncount=ncount+nclust
end do
c
do i=1,nall
do j=i+1,nall
c
prop=real(ind(i,j))/sel(i)
if(prop.ge.cutpair.and.c(i).ge.cpair)then
write(nt,‘(“Freq. for genes:”,2i6,3f12.5)’)
c * ”Single frequencies:”9x,2f12.5)’)
* i,j,prop,c(i),c(j)
end if
end do
end do
end if
c
if(ntupw.gt.0) then
call HROUT(0,ICYCLE,’ ’)
call HREND(‘ani98’)
end if
STOP
END

### EXAMPLE 4

### Microarray Expression Analysis Using Cells from Two Colon Cancer Cell Lines

HT29 cells represent advanced highly aggressive colon tumors. They contain mutations in both the APC gene and p53 gene, to tumor suppressor genes that frequently mutate during colon tumorigenesis HCT116 cells manifest less aggressive colon tumors and harbor functional p53 and APC. They are defective in DNA repair. The experiment was performed with three RNA samples (1 μg RNA each). Cy-3-dCTP (green) was used to label HCT 116 cells while Cy-5dCTP (red) was used for HT29 cells. Each comparison set was hybridized against two microarray slides (facing each other) containing 4608 minimally redundant cDNAs spotted in duplicate. As control, six Drosophila genes were added to the Cy-5 samples. Thus, in a red vs. green comparison they are differentially expressed by design. The experiment resulted in a total of twelve measurements on each channel for each gene on the microarrays. Although a nested dependence structure existed in the samples, the analysis assumes them as independent replicates. Additionally, ten HCT116 samples hybridized with Cy-5 (red) from a separate experiment were included in the analysis.

Two comparisons were performed: (i) HCT116 vs. HT29 and (ii) HCT116 (green) vs. HCT116 (red); the first is inter cell lines whereas the second is intra cell lines. The relevant parameters for the random search were set: N_{cycle}=10,000, N_{subset}=5; and, the Mahalanobis distance was used as the quality function.

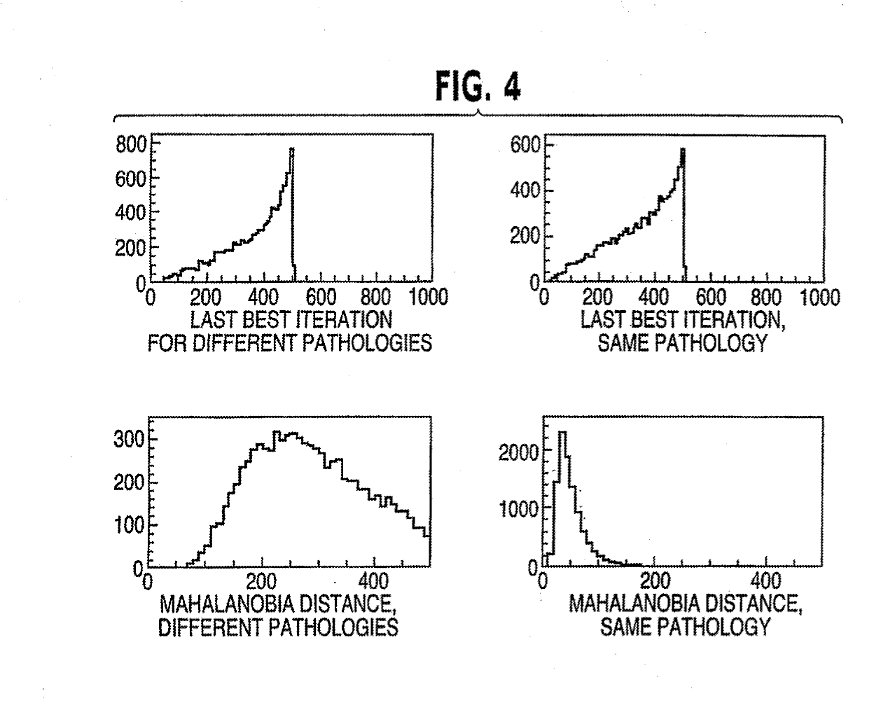

Referring to FIG. 4, the left panel corresponds to the comparison of the different cell lines (as the case (i) above) whereas the right panel to the comparison of the same cell line on different channels (as the case (ii) above). The histograms of the last best iteration (the top two graphs) are very similar in both cases; neither has reached the global maximum. That is, in both cases, the procedure kept exploring the local maxima due to the early stopping. However, turning to the bottom two graphs, the distribution of the estimated Mahalanobis distances at these local maxima in each case is very different from each other: When different cell lines were compared, i.e., in the case (i) above, the Mahalanobis distances based on the locally optimal subsets tended to be much larger than those in the case (ii) above when the same cell lines were compared. Therefore, the separation of the two tissues was considerably better in case (i) than in case (ii), as one would expect.

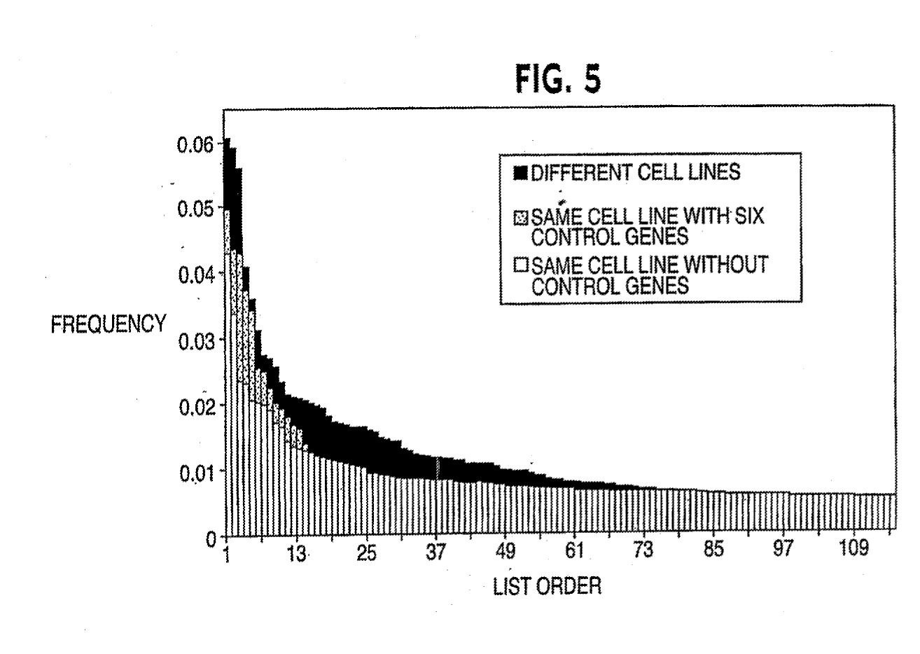

Referring to FIG. 5, the first 115 genes ordered according to the decreasing frequency of occurrence in the selected subsets are plotted. The white columns represent genes from same cell line samples without control whereas the black columns represent genes from different cell line samples. In addition, the gray columns represent genes from same cell lines samples with control. As shown, the right tails of the histograms are very close to each other. Some of the genes in the HCT116/HT29 comparison (the black columns) are selected more often—i.e., have higher frequency—than expected under the null hypothesis of no difference between the two tissues (the white columns). Interestingly, in the case with same cell line without control (the white columns), only two genes had a frequency that was higher than 3%; and, when the control genes were included (the gray columns), this number increased to six and four out of the top five genes (Nos. 1, 2, 3, and 5 on the x axis) were actually Drosophila control genes.

A frequency level of 1% was selected as the cutoff for identifying differentially expressed genes. Total 59 genes were selected and thus 59 cDNA spots were identified on the slides. A comparison was carried out between the 59 cDNA spots and the top 59 genes selected by t-statistic. Almost half of those genes (25 to be exact) were identified by both methods. However, a characteristic advantage of the multivariate random search procedure was its ability to identify correlated genes. Some of the genes had several corresponding spots on the slides, and therefore their expression levels at various spots were known to be correlated. Among the 59 genes identified by the multivariate random search method, 13 had two, and two had three spots inter-related to each other. By comparison, among the genes identified by the marginal t-statistic, 17 genes had two or more replicates on the slides, and only one of them had all of its replicates selected in the resulting list of genes. Therefore, the improved random search procedure of this invention is powerful in identifying less pronounced differentially expressed genes when they are correlated with more strongly differentially expressed genes.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A method for identifying a set of genes from a multiplicity of genes whose expression levels at a first state and a second state are measured in replicates using one or more, nucleotide arrays, thereby generating a first plurality of independent measurements of the expression levels for said first state and a second plurality of independent measurements of the expression levels for said second state, which method comprises:
(a) identifying a quality function capable of evaluating the distinctiveness between the first plurality and the second plurality;
(b) selecting a subset of genes, whose expression levels in said first state and second state are represented in said first plurality and said second plurality, respectively;
(c) calculating the values of the quality function for said subset of genes in said first state and said second state based on the first and second plurality, thereby determining the distinctiveness of the first and the second plurality;
(d) substituting a gene in said subset with one outside of said subset, thereby generating a new subset and repeating step (c), keeping the new subset if the distinctiveness increases and the original subset if otherwise;
(e) repeating steps (c) and (d) for a first predetermined number of times, thereby identifying a locally optimal subset of genes;
(f) repeating steps (b) to (e) for a second predetermined number of times, thereby identifying said second predetermined number of the locally optimal subsets; and
(g) integrating said second predetermined number of the locally optimal subsets into said set of genes, wherein said set is larger than said locally optimal subsets in size.

2. The method of claim 1 wherein said states are selected from the group consisting of biological states physiological states, pathological states, and prognostic states.

3. A method for identifying a set of genes from a multiplicity of genes whose expression levels at a first tissue and a second tissue are measured in replicates using one or more nucleotide arrays, thereby generating a first plurality of independent measurements of the expression levels for said first tissue and a second plurality of independent measurements of the expression levels for said second tissue, which method comprises:
(a) identifying a quality function capable of evaluating the distinctiveness between the first plurality and the second plurality;
(b) selecting a subset of genes, whose expression levels in said first tissue and second tissue are represented in said first plurality and said second plurality, respectively;
(c) calculating the values of the quality function for said subset of genes in said first tissue and second tissue based on the first and second plurality, thereby determining the distinctiveness of the first and the second plurality;
(d) substituting a gene in said subset with one outside of said subset, thereby generating a new subset, and repeating step (c), keeping the new subset if the distinctiveness increases and the original subset if otherwise;
(e) repeating steps (c) and (d) for a first predetermined number of times, thereby identifying a locally optimal subset of genes;
(f) repeating steps (b) to (e) for a second predetermined number of times, thereby identifying said second predetermined number of the locally optimal subsets; and
(g) integrating said second predetermined number of the locally optimal subsets into said set of genes, wherein said set is larger than said locally optimal subsets in size.

4. The method of claim 3, wherein said tissues are selected from the group consisting of normal lung tissues, cancer lung tissues, normal heart tissues, pathological heart tissues, normal and abnormal colon tissues, normal and abnormal renal tissues, normal and abnormal prostate tissues, and normal and abnormal breast tissues.

5. A method for identifying a set of genes from a multiplicity of genes whose expression levels in a first type of cells and a second type of cells are measured in replicates using one or more nucleotide arrays, thereby generating a first plurality of independent measurements of the expression levels for said first type of cells and a second plurality of independent measurements of the expression levels for said second type of cells, which method comprises:
(a) identifying a quality function capable of evaluating the distinctiveness between the first plurality and the second plurality;
(b) selecting a subset of genes, whose expression levels in said first type of cells and said second type of cells are represented in said first plurality and said second plurality, respectively;
(c) calculating the values of the quality function for said subset of genes in said first type of cells and said second type of cells based on the first and second plurality, thereby determining the distinctiveness of the first and the second plurality;
(d) substituting a gene in said subset with one outside of said subset, thereby generating a new subset, and repeating step (c), keeping the new subset if the distinctiveness increases and the original subset if otherwise;
(e) repeating steps (c) and (d) for a first predetermined number of times, thereby identifying a locally optimal subset of genes;
(f) repeating steps (b) to (e) for a second predetermined number of times, thereby identifying said second predetermined number of the locally optimal subsets; and
(g) integrating said second predetermined number of the locally optimal subsets into said set of genes, wherein said set is larger than said locally optimal subsets in size.

6. The method of claim 5, wherein said types of cells are selected from the group consisting of normal lung cells, cancer lung cells, normal heart cells, pathological heart cells, normal and abnormal colon cells, normal and abnormal renal cells, normal and abnormal prostate cells, and normal and abnormal breast cells.

7. The method of claim 5, wherein said type of cells are selected from the group consisting of cultured cells and cells isolated from an organism.

8. The method of claim 1, 3, or 5, wherein said integrating is performed by selecting the genes whose frequency of occurrences in said second predetermined number of the final subsets exceeds a third predetermined number.

9. The method of claim 8, wherein said third predetermined number is 1% or 5%.

10. The method of claim 1, 3, or 5, wherein said first predetermined number is sufficiently small such that the global maximum is not reached.

11. The method of claim 1, 3, or 5, wherein said quality function is a parametric function or a non-parametric function.

12. The method of claim 11, wherein said parametric function is selected from the group consisting of the Mahalanobis distance and the Bhattacharya distance.

13. The method of claim 1, 3, or 5, wherein the nucleotide arrays are selected from the group consisting of arrays having spotted thereon cDNA sequences and arrays having synthesized thereon oligonucleotides.
